SEQUENCE LISTING (28 sequences)

SEQ ID NO: 1 - DNA, 24 bp, Artificial Sequence
  Synthetic DNA primer
cgtttttgccttctgacttctttc

SEQ ID NO: 2 - DNA, 21 bp, Artificial Sequence
  Synthetic DNA primer
acagagctgaggcggtgtcta

SEQ ID NO: 3 - DNA, 21 bp, Artificial Sequence
  Synthetic DNA primer
ccgtctgtgccttctcatctg

SEQ ID NO: 4 - DNA, 29 bp, Artificial Sequence
  Synthetic DNA primer
agtccaagagtcctcttatgtaagacctt

SEQ ID NO: 5 - DNA, 21 bp, Artificial Sequence
  Silencer select human siRNA sequences
gcgcauuccuuugcaagcatt

SEQ ID NO: 6 - DNA, 21 bp, Artificial Sequence
  Silencer select human siRNA sequences
ugcuugcaaaggaaugcgctg

SEQ ID NO: 7 - DNA, 21 bp, Artificial Sequence
  Silencer select human siRNA sequences
gagucuaccuaauucuggatt

SEQ ID NO: 8 - DNA, 21 bp, Artificial Sequence
  Silencer select human siRNA sequences
uccagaauuagguagacuctg

SEQ ID NO: 9 - DNA, 21 bp, Artificial Sequence
  Silencer select human siRNA sequences
ggaucagcuggagagcuuatt

SEQ ID NO: 10 - DNA, 21 bp, Artificial Sequence
  Silencer select human siRNA sequences
uaagcucuccagcugauccaa

SEQ ID NO: 11 - DNA, 21 bp, Artificial Sequence
  Silencer select human siRNA sequences
ccugcgucucuacaacuautt

SEQ ID NO: 12 - DNA, 21 bp, Artificial Sequence
  Silencer select human siRNA sequences
auaguuguagagacgcaggat

SEQ ID NO: 13 - DNA, 21 bp, Artificial Sequence
  Silencer select human siRNA sequences
ucgucaagguggaccuaaatt

SEQ ID NO: 14 - DNA, 21 bp, Artificial Sequence
  Silencer select human siRNA sequences
uuuagguccaccuugacgatg

SEQ ID NO: 15 - DNA, 21 bp, Artificial Sequence
  Silencer select human siRNA sequences
gcaaguuuggaaacguguatt

SEQ ID NO: 16 - DNA, 21 bp, Artificial Sequence
  Silencer select human siRNA sequences
uacacguuuccaaacuugcct

SEQ ID NO: 17 - DNA, 21 bp, Artificial Sequence
  Silencer select human siRNA sequences
ccgugagaguuccaacuuutt

SEQ ID NO: 18 - DNA, 21 bp, Artificial Sequence
  Silencer select human siRNA sequences
aaaguuggaacucucacggtg

SEQ ID NO: 19 - DNA, 21 bp, Artificial Sequence
  Silencer select human siRNA sequences
caguuuaucugcauccgaatt

SEQ ID NO: 20 - DNA, 21 bp, Artificial Sequence
  Silencer select human siRNA sequences
uucggaugcagauaaacugag

SEQ ID NO: 21 - DNA, 21 bp, Artificial Sequence
  Silencer select human siRNA sequences
ggaggguccuuaucguagatt

SEQ ID NO: 22 - DNA, 21 bp, Artificial Sequence
  Silencer select human siRNA sequences
ucuacgauaaggacccuccgg

SEQ ID NO: 23 - DNA, 21 bp, Artificial Sequence
  Silencer select human siRNA sequences
gaaggucuccgaugucauatt

SEQ ID NO: 24 - DNA, 21 bp, Artificial Sequence
  Silencer select human siRNA sequences
uaugacaucggagaccuuctt

SEQ ID NO: 25 - DNA, 21 bp, Artificial Sequence
  Silencer select human siRNA sequences
gaccuacuaucgggaugaatt

SEQ ID NO: 26 - DNA, 21 bp, Artificial Sequence
  Silencer select human siRNA sequences
uucaucccgauaguagguctt

SEQ ID NO: 27 - DNA, 21 bp, Artificial Sequence
  Silencer select human siRNA sequences
ccauugaccugcacaccgatt

SEQ ID NO: 28 - DNA, 21 bp, Artificial Sequence
  Silencer select human siRNA sequences
ucggugugcaggucaauggaa

(19) United States
(12) Patent Application Publication
(10) Pub. No.: US 2022/0288102 A1
(43) Pub. Date: Sep. 15, 2022

(54) Compounds for Hepatitis B Treatment

(71) Applicant: Agency for Science, Technology and Research, Singapore (SG)

(72) Inventors: Giridharan Periyasamy, Genome (SG); Ramanuj Dasgupta, Genome (SG); Narmada Balakrishnan Chakra-Pani, Genome (SG)

(21) Appl. No.: 17/633,778

(22) PCT Filed: Aug. 11, 2020
(86) PCT No.: PCT/SG2020/050462

§ 371 Date: Feb. 8, 2022

(30) Foreign Application Priority Data
Aug. 8, 2019 (SG) 10201907372T

(51) Int. Cl. A61K 31/713; A61K 45/06; A61P 31/20

(52) U.S. Cl. CPC A61K 31/713; A61K 45/06; A61P 31/20

(57) ABSTRACT

Described herein is a compound selected from the group consisting of AZ960, CYC116, MI-3, Nexturastat A, TAK-901, Tubastatin A hydrochloride salt, and a small interfering RNA molecule comprising any one of SEQ ID No. 5 to SEQ ID No. 28 for use in the treatment of Hepatitis B. The compound may also be used in the manufacture of a medicament for the treatment of Hepatitis B. Also described is a method of treating a Hepatitis B infection in a subject, the method comprises administering to the subject a therapeutically effective dose of the compound as described above.

The present invention is related to new methods and compounds for the treatment of Hepatitis B.

Viral hepatitis is inflammation of the liver caused by a viral infection. There are five main Hepatitis viruses A-E which causes hepatitis. Of these Hepatitis B and Hepatitis C often cause the most serious conditions in patients. The Hepatitis B virus (HBV) can cause both acute and chronic hepatitis when the infected patient is unable to completely eliminate the virus after infection. Chronic hepatitis can lead to cirrhosis or hepatocellular carcinoma (a type of liver cancer) in the patient.

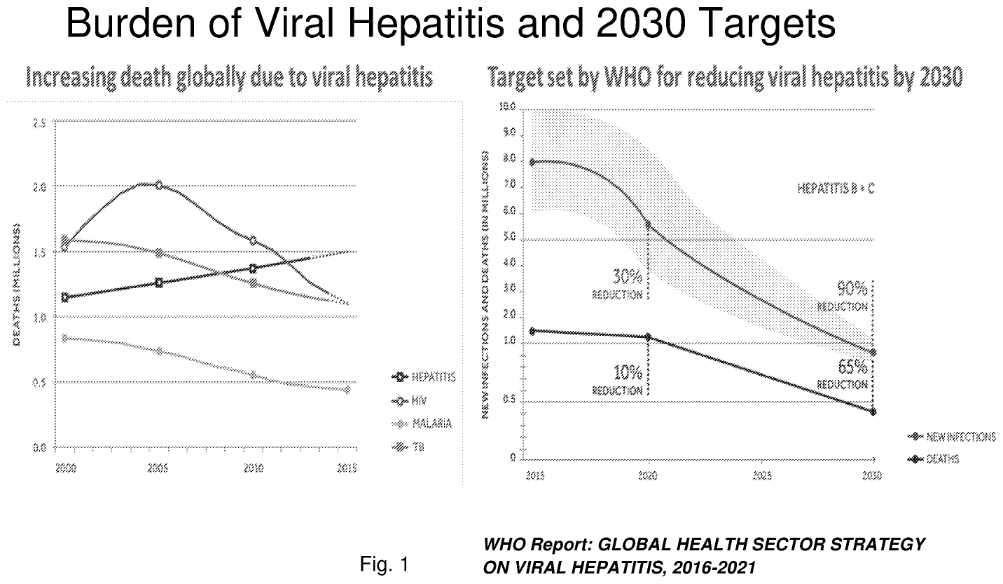

Although vaccines are available to prevent Hepatitis A and B, and an effective but costly treatment for Hepatitis C, the global deaths attributed to hepatitis is on an increasing trend as shown in FIG. 1. In contrast, other viral diseases like HIV, malaria and tuberculosis (TB) are on a definite downward trend. The World Health Organisation (WHO) has set a target of reducing new infections of Hepatitis B and C and deaths by 90% and 65% respectively by 2030. In particular, this may be by identifying a functional cure for Hepatitis B as a standard of care drug is available for Hepatitis C.

Hence, there is a need for new treatment methods and drugs for hepatitis in particular Hepatitis B. One method is to prepare new compounds and determine its effectiveness against the disease. Another method is to repurpose existing compounds, including compounds that had failed to advance from clinical trials to approved drugs on the market. Finding new indications for existing drugs is particularly attractive as the potential side effects are usually better known, and adding new indications to an existing drug is often easier to obtain regulatory approval. This similarly applies to compounds which may have not reached the market due to a lack of efficacy alone or in comparison with a standard of care approved drug.

In an aspect of the invention, there is provided a compound selected from the group consisting of AZ960, CYC116, MI-3, Nexturastat A, TAK-901, Tubastatin A hydrochloride salt, and a small interfering RNA molecule comprising any one of SEQ ID No. 5 to SEQ ID No. 28 for use in the treatment of Hepatitis B.

In another aspect of the invention, there is provided use of a compound selected from the group consisting of AZ960, CYC116, MI-3, Nexturastat A, TAK-901, Tubastatin A hydrochloride salt, and a small interfering RNA molecule comprising any one of SEQ ID No. 5 to SEQ ID No. 28 in the manufacture of a medicament for the treatment of Hepatitis B.

The inhibitors above include any pharmaceutically acceptable salt form and/or prodrug from the of the inhibitor. Tubastatin A hydrochloride salt is already a salt but other pharmaceutically acceptable salt may be used.

The phrase SEQ ID No. 5 to SEQ ID No. 28 refers to SEQ ID No.5, SEQ ID No. 6, SEQ ID No. 7, SEQ ID No. 8, SEQ ID No. 9, SEQ ID No. 10, SEQ ID No. 11, SEQ ID No. 12, SEQ ID No. 13, SEQ ID No. 14. SEQ ID No. 15, SEQ ID No. 16, SEQ ID No. 17, SEQ ID No. 18, SEQ ID No. 19, SEQ ID No. 20, SEQ ID No. 21, SEQ ID No. 22, SEQ ID No. 23, SEQ ID No. 24, SEQ ID No. 25, SEQ ID No. 26, SEQ ID No. 27, and SEQ ID No. 28. This is similarly applicable to any such similar phrases used herein.

The menin-MLL interaction may be disrupted by menin inhibitors as reported by Cierpicki and Gremecka (Future Med. Chem. 2014, 6(4), 447-462) as menin binds to both MLL1 and MLL2 and has a protein binding site with well defined architecture, rigid in structure and without conformation change upon binding of protein ligands. Thus, a menin inhibitor can function as a menin-MLL inhibitor.

In both of the aspects above, the compound may be preferably selected from the group consisting of CYC116, Nexturastat A, TAK-901, Tubastatin A hydrochloride salt, and a small interfering RNA molecule comprising any one of SEQ ID No. 5 to SEQ ID No. 22.

In another aspect of the invention, there is provided a small interfering RNA molecule comprising any one of SEQ ID No. 5 to SEQ ID No. 28. Preferably, the small interfering RNA molecule is for use in the treatment of Hepatitis B or used in the manufacture of a medicament for the treatment of Hepatitis B.

In another aspect of the invention, there is provided a method of treating a Hepatitis B infection in a subject, the method comprises administering to the subject a therapeutically effective dose of a compound selected from the group consisting of AZ960, CYC116, MI-3, Nexturastat A, TAK-901, Tubastatin A hydrochloride salt, and a small interfering RNA molecule comprising any one of SEQ ID No. 5 to SEQ ID No. 28,

Preferably, the compound is selected from the group consisting of CYC116, Nexturastat A, TAK-901, Tubastatin A hydrochloride salt, and a small interfering RNA molecule comprising any one of SEQ ID No. 5 to SEQ ID No. 22.

Preferably, the method further comprises administering a nucleoside analogue or pegylated interferon active against a Hepatitis B virus.

Preferably, the nucleoside analogue is selected from the group consisting of entecavir, tenofovir, lamivudine, adefovir, telbivudine, and any prodrug and/or any pharmaceutically salt form of the compounds. The pegylated interferon may be pegylated interferon alfa-2a, or may be pegylated interferon alfa-2a, pegylated interferon alfa-2b and pegylated interferon beta-1a.

In another aspect of the invention, there is provided a composition comprising a compound selected from the group selected from the group consisting of AZ960, CYC116, MI-3, Nexturastat A, TAK-901, Tubastatin A hydrochloride salt, and a small interfering RNA molecule comprising any one of SEQ ID No. 5 to SEQ ID No. 28; and a nucleoside analogue or pegylated interferon active against a Hepatitis B virus. The pegylated interferon may be pegylated interferon alfa-2a, or may be pegylated interferon alfa-2a, pegylated interferon alfa-2b and pegylated interferon beta-1a.

Preferably, the compound is selected from the group consisting of CYC116, Nexturastat A, TAK-901, Tubastatin A hydrochloride salt, and a small interfering RNA molecule comprising any one of SEQ ID No. 5 to SEQ ID No. 22.

Preferably, the nucleoside analogue is selected from the group consisting of entecavir, tenofovir, lamivudine, adefovir, telbivudine, and any prodrug and/or any pharmaceutically salt form of the compounds.

In another aspect of the invention, the composition above is for use as a medicament.

Preferably, the composition is for use in the treatment of Hepatitis B.

Preferably, the composition is used in the manufacture of a medicament for the treatment of Hepatitis B.

In the Figures:

FIG. 1 shows the burden of viral hepatitis and the WHO's 2030 target to reduce hepatitis infections and deaths;

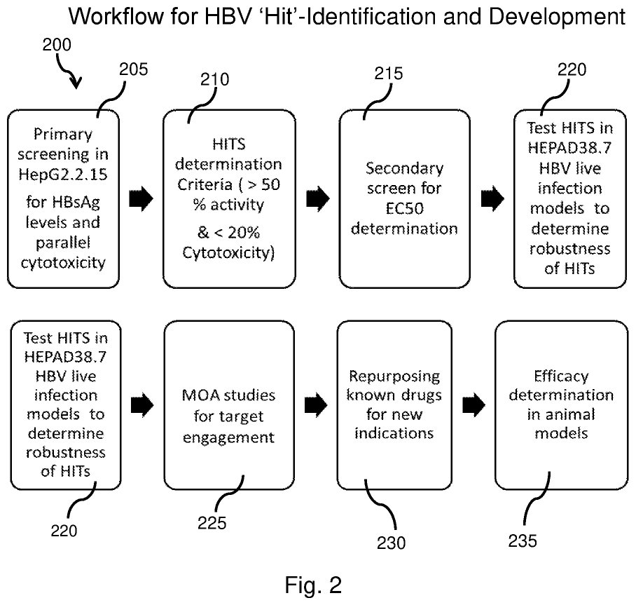

FIG. 2 shows a method for identifying active compounds (“HIT”) against HBV;

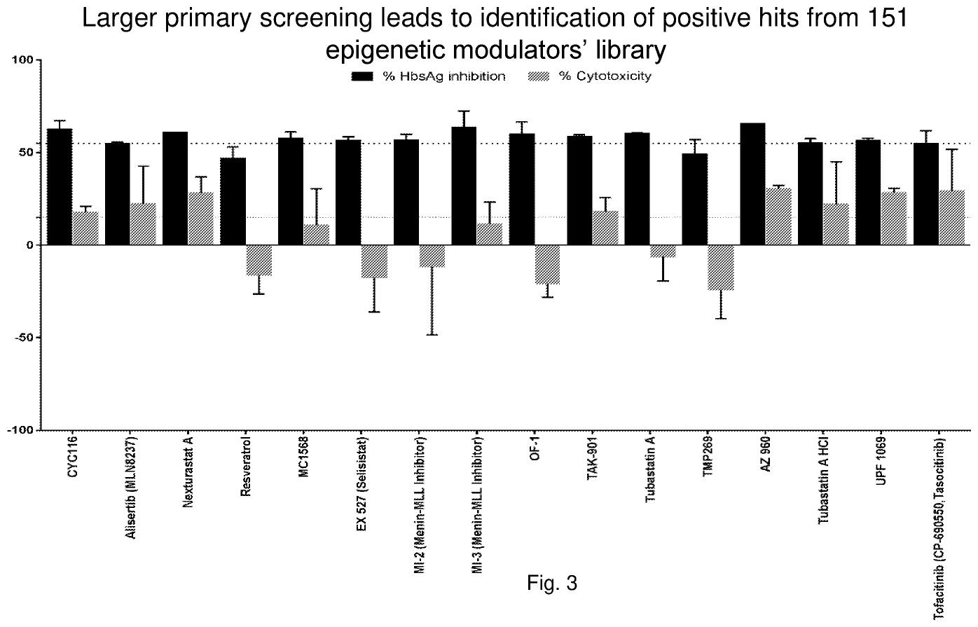

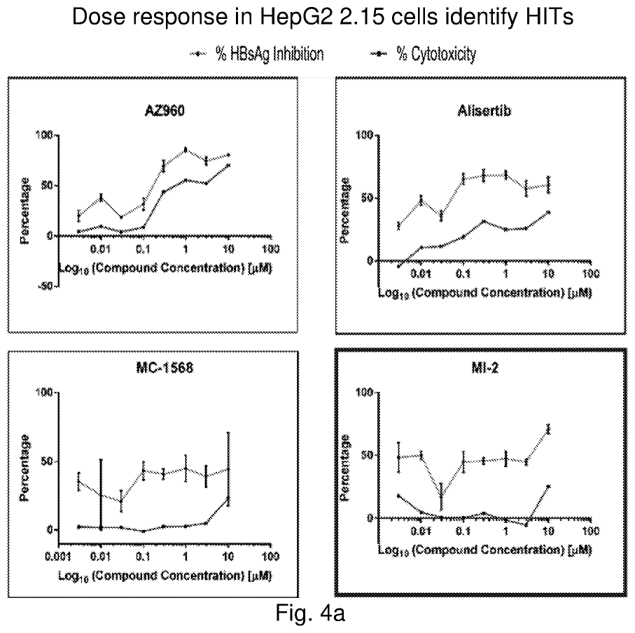

FIG. 3 shows the inhibition and cytotoxicity results of the HIT compounds identified from a epigenetic modulators' library;

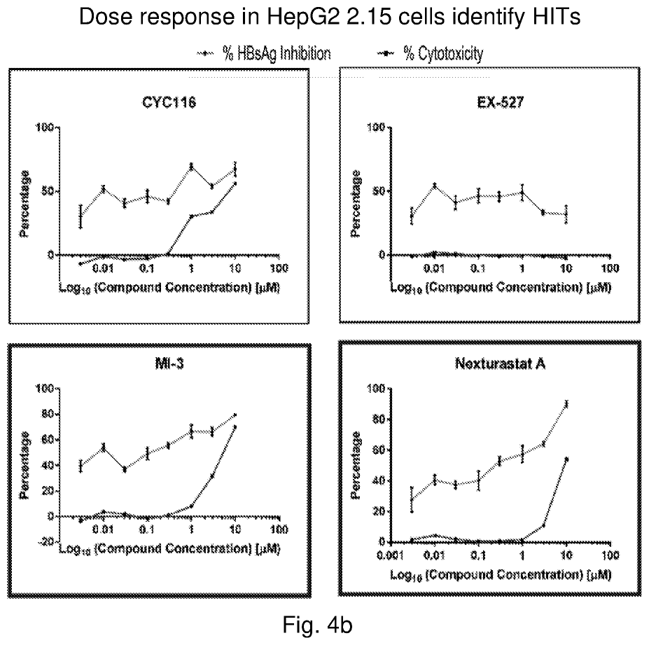

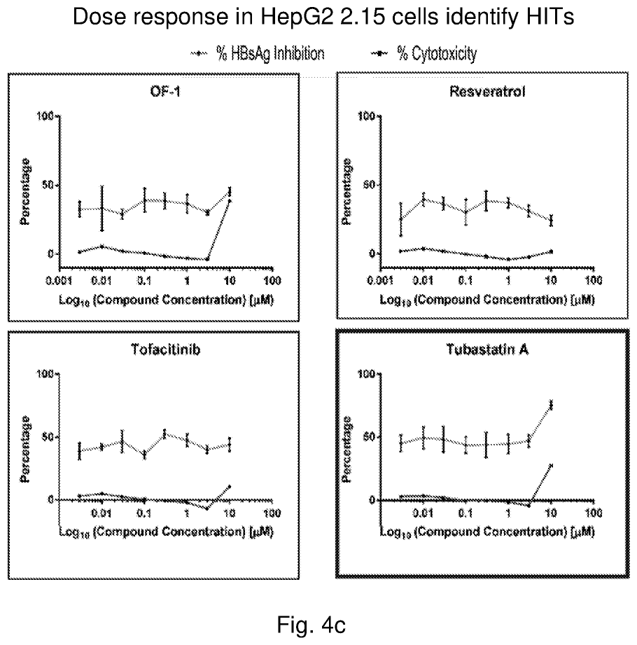

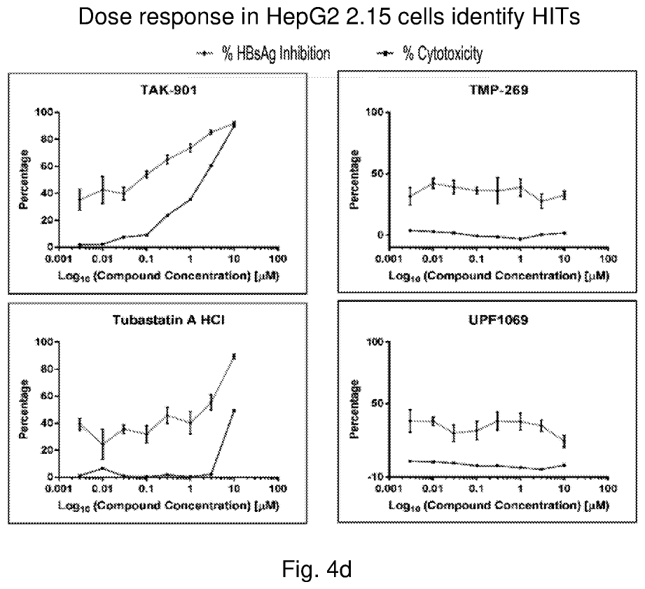

FIG. 4 shows the dose response curves of the 16 HIT compounds in HepG2 2.15 cells;

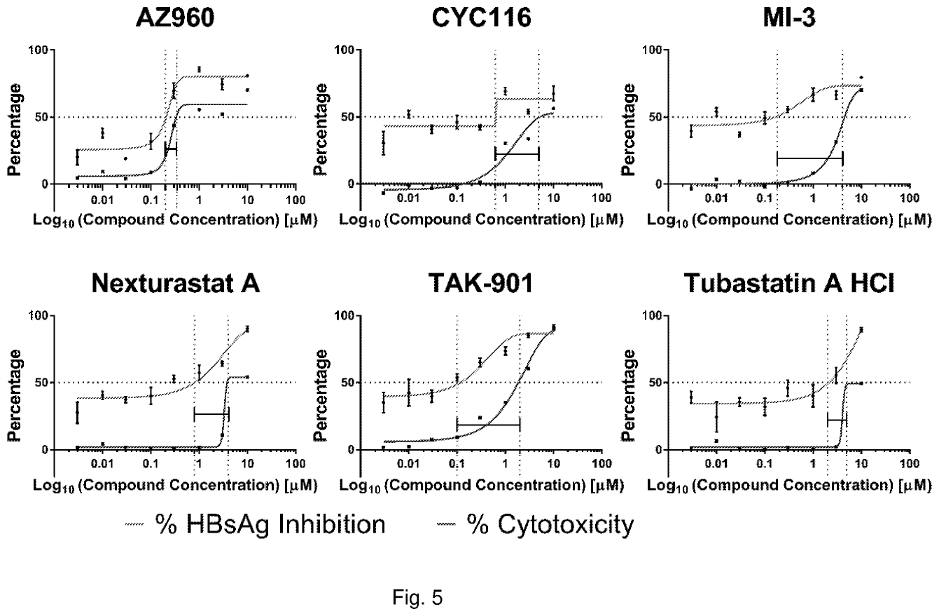

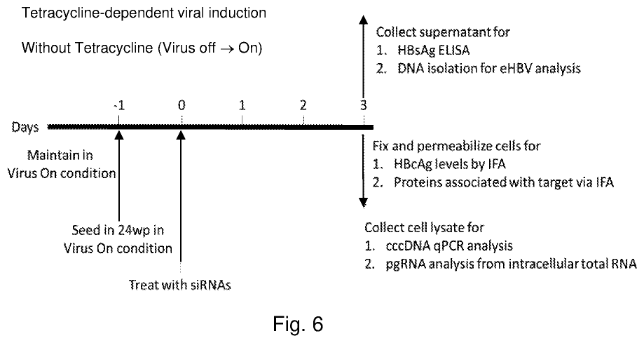

FIG. 5 shows the 6 selected HITs and their therapeutic window FIG. 6 shows the conditions used to culture HepAD38.7 cells (siRNA);

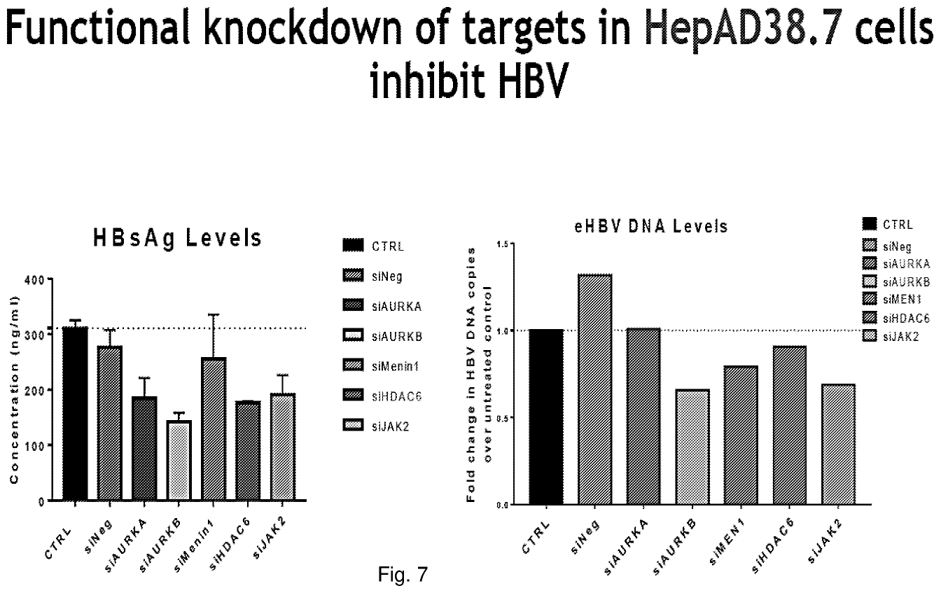

FIG. 7 shows the results of the functional knockdown of targets in HepAD38.7 cells inhibit HBV;

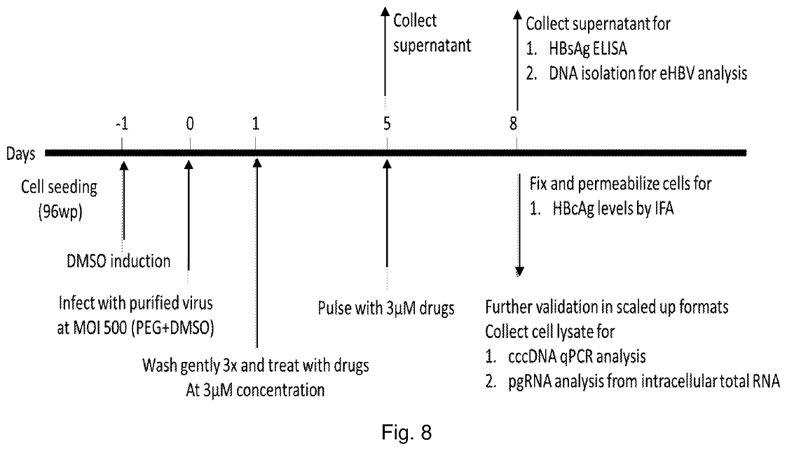

FIG. 8 shows the conditions used to introduce HBV infection and drug treatment schedule in primary human hepatocytes;

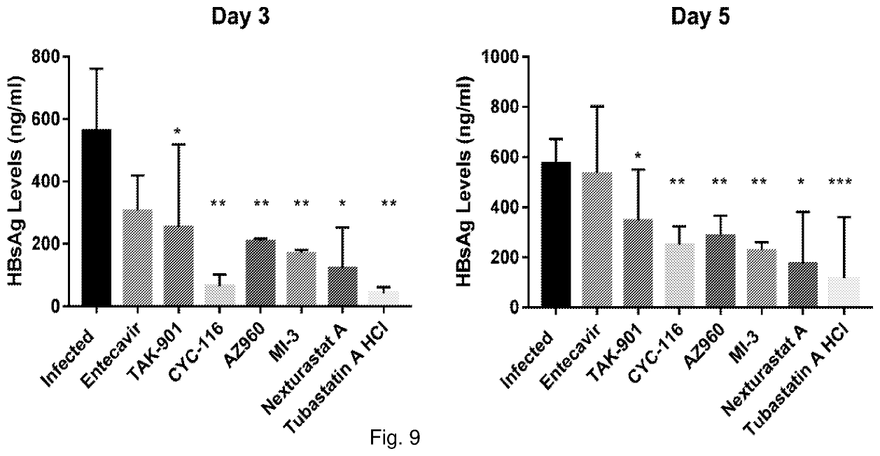

FIG. 9 shows that the HIT compounds reduce HBsAg in infected primary human hepatocytes;

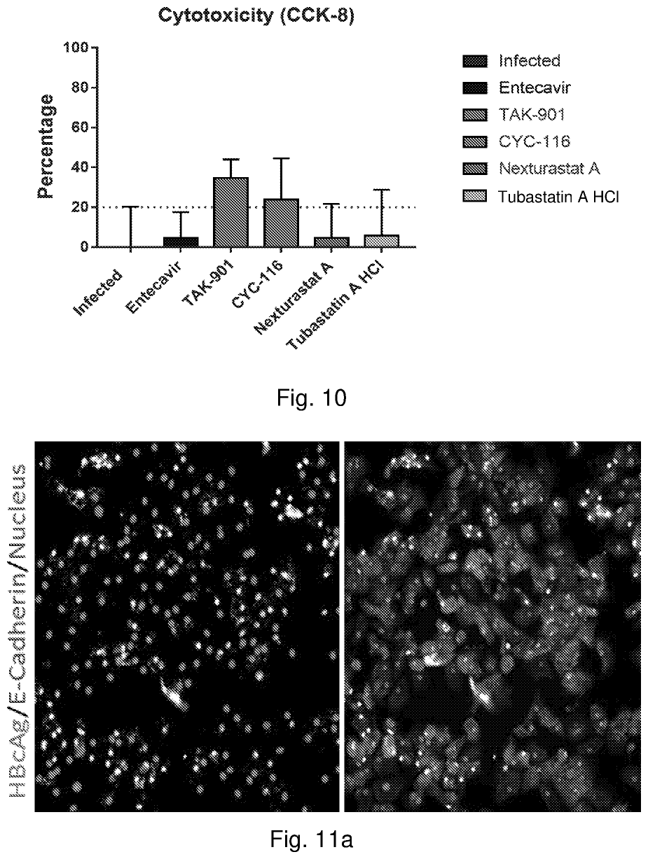

FIG. 10 shows that the HITs have low cytotoxicity in infected primary human hepatocytes

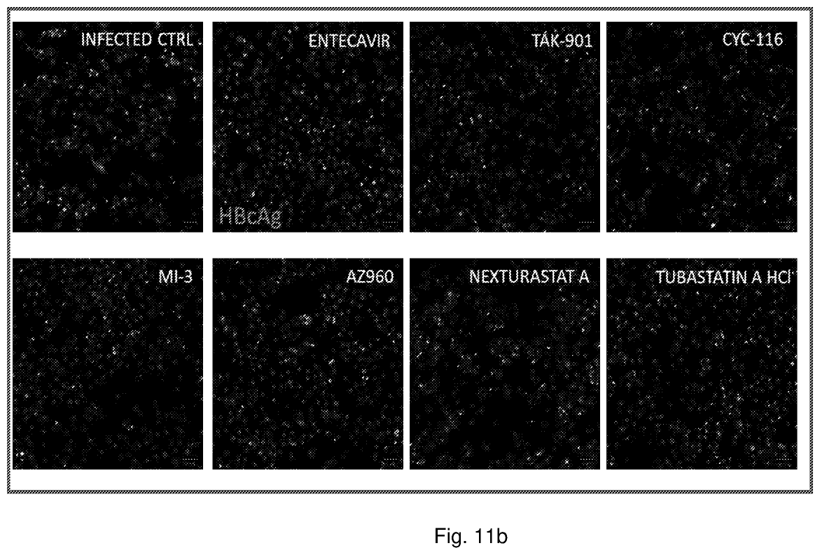

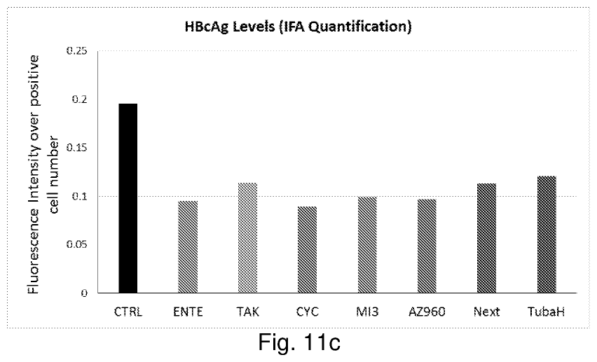

FIG. 11 shows the immunofluorescence images after the treatment of infected primary human hepatocytes with the HIT compounds, FIG. 11a shows the HBcAg levels in the infected control with E-cadherin in the background, FIG. 11b. shows the HBcAg levels in the drug-treated cells at day 8, and FIG. 11c shows the quantification data of HBcAg intensity in FIG. 11b;

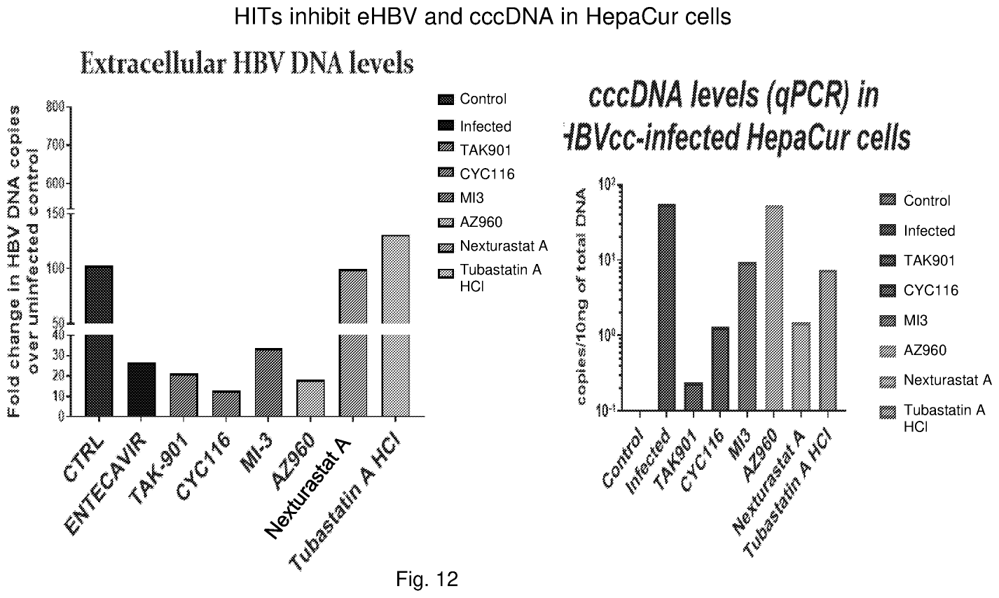

FIG. 12 shows that the HIT compounds inhibit eHBV and cccDNA levels in primary human hepatocytes;

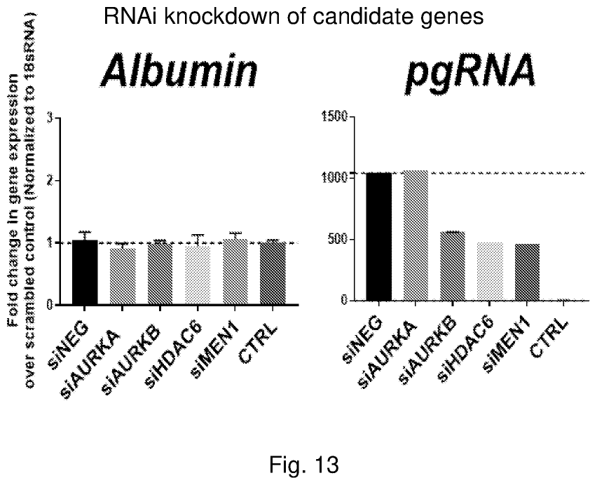

FIG. 13 shows the effects of the RNAi knockdown of candidate genes on albumin and pgRNA levels;

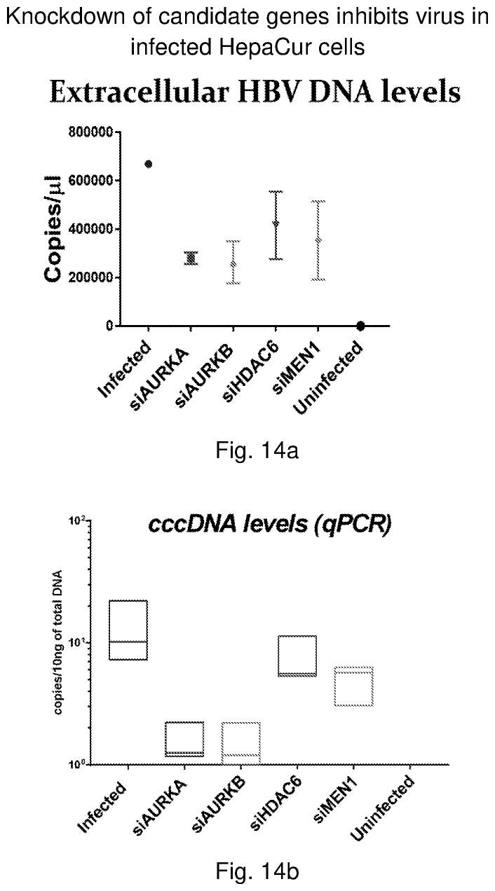

FIG. 14 shows that the knockdown of candidate genes inhibits virus in infected primary human hepatocytes, FIGS. 14a and 14b respectively show the extracellular HBV DNA levels (in cell culture supernatant) and cccDNA levels measured by qPCR.

In the following description, numerous specific details are set forth in order to provide a thorough understanding of various illustrative embodiments of the invention. It will be understood, however, to one skilled in the art, that embodiments of the invention may be practiced without some or all of these specific details. Embodiments described in the context of one of the methods or devices are analogously valid for the other methods or devices. Similarly, embodiments described in the context of a method are analogously valid for a device, and vice versa.

Features that are described in the context of an embodiment may correspondingly be applicable to the same or similar features in the other embodiments. Features that are described in the context of an embodiment may correspondingly be applicable to the other embodiments, even if not explicitly described in these other embodiments. Furthermore, additions and/or combinations and/or alternatives as described for a feature in the context of an embodiment may correspondingly be applicable to the same or similar feature in the other embodiments.

The terms “about”, “approximately”, “substantially” must be read with reference to the context of the application as a whole, and have regard to the meaning a particular technical term qualified by such a word usually has in the field concerned. For example, it may be understood that a certain parameter, function, effect, or result can be performed or obtained within a certain tolerance, and the skilled person in the relevant technical field knows how to obtain the tolerance of such term.

The phrase “at least one of A and B” means it requires only A alone, B alone, or A and B, i.e. only one of A or B is required. The phrase “A and/or B” includes A alone, B alone and A and B.

The term “agent” and “drug” are used herein, for purposes of the specification and claims, to mean chemical compounds, mixtures of chemical compounds, biological macromolecules, or extracts made from biological materials such as bacteria, plants, fungi, or animal particularly mammalian) cells or tissues that are suspected of having therapeutic properties. The agent or drug may be purified, substantially purified or partially purified.

The term “morphology” is used herein, for purposes of the specification and claims, to mean the visual appearance of a cell or organism when viewed with the eye, a light microscope, a confocal microscope or an electron microscope, as appropriate.

The term “subject,” “individual,” and “patient” are used herein, for purposes of the specification and claims, to mean a human or other animal, such as farm animals or laboratory animals (e.g., guinea pig or mice) capable of having cell cycle (influenced) determined diseases, either naturally occurring or induced, including but not limited to cancer.

The term “synergistic effect” as used herein means the combined effect of two or more anticancer agents or chemotherapy drugs can be greater than the sum of the separate effects of the anticancer agents or chemotherapy drugs alone.

The term “therapeutically effective amount” means the amount of the subject compound that will elicit a desired response, for example, a biological or medical response of a tissue, system, animal, or human that is sought, for example, by a researcher, veterinarian, medical doctor, or other clinician.

Throughout the specification, any recitation of a particular compound should be understood to encompass that compound, prodrug of the compound and any (other) pharmaceutically acceptable salts thereof. The term “pharmaceutically acceptable salt” refers to a salt of a compound that does not cause significant irritation to an organism to which it is administered and does not abrogate the biological activity and properties of the compound. In some embodiments, the salt is an acid addition salt of the compound. Pharmaceutical salts can be obtained by reacting a compound with inorganic acids such as hydrohalic acid (e.g., hydrochloric acid or hydrobromic acid), sulfuric acid, nitric acid, phosphoric acid and the like. Pharmaceutical salts can also be obtained by reacting a compound with an organic acid such as aliphatic or aromatic carboxylic or sulfonic acids, for example acetic, succinic, lactic, malic, tartaric, citric, ascorbic, nicotinic, methanesulfonic, ethanesulfonic, camphorsulfonic, p-toluenesulfonic, salicylic or naphthalenesulfonic acid. Pharmaceutical salts can also be obtained by reacting a compound with a base to form a salt such as an ammonium salt, an alkali metal salt, such as a sodium or a potassium salt, an alkaline earth metal salt, such as a calcium or a magnesium salt, a salt of organic bases such as dicyclohexylamine, N-methyl-D-glucamine, tris(hydroxymethyl)methylamine, C1-C7 alkylamine, cyclohexylamine, triethanolamine, ethylenediamine, and salts with amino acids such as arginine, lysine, and the like.

As used herein, “activity” or “biological activity” of a polypeptide refers to any biological function or any biological interaction of a polypeptide. Activity of a polypeptide may refer to the polypeptide's enzymatic or catalytic activity. Activity of a polypeptide may also refer to the polypeptide's binding with another polypeptide, a polynucleotide, or other agents in a cell.

As used herein, “EC50,” is intended to refer to the concentration of a substance (e.g., a compound or a drug) that is required for 50% agonism or activation of a biological process, or component of a process, including a protein, subunit, organelle, ribonucleoprotein, etc. In one aspect, an EC50 can refer to the concentration of a substance that is required for 50% agonism or activation in vivo, as further defined elsewhere herein. In a further aspect, EC50 refers to the concentration of agonist or activator that provokes a response halfway between the baseline and maximum response.

As used herein, “IC50,” is intended to refer to the concentration of a substance (e.g., a compound or a drug) that is required for 50% inhibition of a biological process, or component of a process, including a protein, subunit, organelle, ribonucleoprotein, etc. For example, an IC50 can refer to the concentration of a substance that is required for 50% inhibition in vivo or the inhibition is measured in vitro, as further defined elsewhere herein. Alternatively, IC50 refers to the half maximal (50%) inhibitory concentration (IC) of a substance The inhibition can be measured in a cell-line such as AN3 CA, BT-20, BT-549, HCT 116, HER218, MCF7, MDA-MB-231, MDA-MB-235, MDA-MB-435S, MDA-MB-468, PANC-1, PC-3, SK-N-MC, T-47D, and U-87 MG. In a yet further aspect, the inhibition is measured in a cell-line, e.g. HEK-293 or HeLa, transfected with a mutant or wild-type mammalian histone demethylase, e.g. LSD1 or LSD2.

The term “analogue” means a molecule that is not identical, but has analogous functional or structural features. For example, a nucleoside analogue mimics a nucleoside activity but is structurally different either in the nucleoside portion or sugar portion. Examples include deoxyadenosine and adenosine analogues, deoxycytidine analogues, guanosine and deoxyguanosine analogues, thymidine and deoxythymidine analogues, and deoxyunidine analogues. For example, a polypeptide analogue retains the biological activity of a corresponding naturally-occurring polypeptide, while having certain biochemical modifications that enhance the analogue's function relative to a naturally occurring polypeptide. Such biochemical modifications could increase the analogue's protease resistance, membrane permeability, or half-life, without altering, for example, ligand binding. An analogue may be a polypeptide with at least one different amino acid from the naturally-occurring polypeptide, and could include an unnatural amino acid.

As used herein, the term “subject” refers to a living organism as a target of administration. The subject of the herein disclosed methods can be a vertebrate, such as a mammal, a fish, a bird, a reptile, or an amphibian. Thus, the subject of the herein disclosed methods can be a human, non-human primate, horse, pig, rabbit, dog, sheep, goat, cow, cat, guinea pig or rodent. The term does not denote a particular age or sex. Thus, adult and newborn subjects, as well as fetuses, whether male or female, are intended to be covered. A patient refers to a subject afflicted with a disease or disorder. The term “patient” includes human and veterinary subjects.

As used herein, the term “prevent” or “preventing” refers to precluding, averting, obviating, forestalling, stopping, or hindering something from happening, especially by advance action. It is understood that where reduce, inhibit or prevent are used herein, unless specifically indicated otherwise, the use of the other two words is also expressly disclosed. In certain aspects, this term can be synonymous with the language “preventative treatment.”

As used herein, the terms “alleviate” or “alleviating” refer to lightening or lessening the severity of a symptom, condition, or disorder. For example, a treatment that reduces the severity of pain in a subject can be said to alleviate pain. It is understood that, in certain circumstances, a treatment can alleviate a symptom or condition without treating the underlying disorder. In certain aspects, this term can be synonymous with the language “palliative treatment.”

As used herein, the terms “administering” and “administration” refer to any method of providing a pharmaceutical preparation to a subject. Such methods are well known to those skilled in the art and include, but are not limited to, oral administration, transdermal administration, administration by inhalation, nasal administration, topical administration, intravaginal administration, ophthalmic administration, intraaural administration, intracerebral administration, rectal administration, and parenteral administration, including injectables such as intravenous administration, intra-arterial administration, intramuscular administration, and subcutaneous administration. Administration can be continuous or intermittent. In various aspects, a preparation can be administered therapeutically; that is, administered to treat an existing disease or condition. In further various aspects, a preparation can be administered prophylactically; that is, administered for prevention of a disease or condition. In one aspect, administration of a tablet refers to oral administration.

In another aspect, the present disclosure relates to a pharmaceutical composition comprising physiologically acceptable surface active agents, carriers, diluents, excipients, smoothing agents, suspension agents, film forming substances, and coating assistants, or a combination thereof; and a compound disclosed herein. The pharmaceutical composition facilitates administration of the compound to an organism. Acceptable carriers or diluents for therapeutic use are well known in the pharmaceutical art, and are described, for example, in Remington's Pharmaceutical Sciences, 18th Ed., Mack Publishing Co., Easton, Pa. (1990), which is incorporated herein by reference in its entirety. Preservatives, stabilizers, dyes, sweeteners, fragrances, flavoring agents, and the like may be provided in the pharmaceutical composition. For example, sodium benzoate, ascorbic acid and esters of p-hydroxybenzoic acid may be added as preservatives. In addition, antioxidants and suspending agents may be used. In various embodiments, alcohols, esters, sulfated aliphatic alcohols, and the like may be used as surface active agents; sucrose, glucose, lactose, starch, crystallized cellulose, mannitol, light anhydrous silicate, magnesium aluminate, magnesium metasilicate aluminate, synthetic aluminum silicate, calcium carbonate, sodium acid carbonate, calcium hydrogen phosphate, calcium carboxymethyl cellulose, and the like may be used as excipients; magnesium stearate, talc, hardened oil and the like may be used as smoothing agents; coconut oil, olive oil, sesame oil, peanut oil, soya may be used as suspension agents or lubricants; cellulose acetate phthalate as a derivative of a carbohydrate such as cellulose or sugar, or methylacetate-methacrylate copolymer as a derivative of polyvinyl may be used as suspension agents; and plasticizers such as ester phthalates and the like may be used as suspension agents.

Additional therapeutic or diagnostic agents may be incorporated into the pharmaceutical compositions. Alternatively or additionally, pharmaceutical compositions may be combined with other compositions that contain other therapeutic or diagnostic agents.

It will be appreciated that compositions provided herein may be in any form which allows for the composition to be administered to a patient. For example, the composition may be in the form of a solid, liquid or gas (e.g., aerosol). Suitable routes of administration include, without limitation, enteral (e.g. oral, or rectal), topical, parenteral (e.g., sublingually, buccally, sublingual, vaginal, or intranasal). The term parenteral as used herein includes subcutaneous injections, intravenous, intraarterial, intradermal, intramuscular, intrasternal, intracavernous, intrathecal, intraperitoneal, intraocular injections or infusion techniques. The pharmaceutical composition is formulated so as to allow the active ingredients contained therein to be bioavailable upon administration of the composition to a patient. Compositions that will be administered to a patient take the form of one or more dosage units, where for example, a tablet may be a single dosage unit, and a container of one or more compounds of the invention in aerosol form may hold a plurality of dosage units. The compounds can also be administered in sustained or controlled release dosage forms, including depot injections, osmotic pumps, pills, transdermal (including electrotransport) patches, and the like, for prolonged and/or timed, pulsed administration at a predetermined rate.

Dosage amount and interval may be adjusted individually to provide plasma levels of the active moiety which are sufficient to maintain the modulating effects, or minimal effective concentration (MEC). The MEC will vary for each compound but can be estimated from in vitro data. Dosages necessary to achieve the MEC will depend on individual characteristics and route of administration. However, HPLC assays or bioassays can be used to determine plasma concentrations. Typically, dosages may be between about 10 microgram/kg and 100 mg/kg body weight, preferably between about 100 microgram/kg and 10 mg/kg body weight. Alternatively dosages may be based and calculated upon the surface area of the patient, as understood by those of skill in the art.

It should be noted that the attending physician would know how to and when to terminate, interrupt, or adjust administration due to toxicity or organ dysfunctions. Conversely, the attending physician would also know to adjust treatment to higher levels if the clinical response were not adequate (precluding toxicity). The magnitude of an administrated dose in the management of the disorder of interest will vary with the severity of the condition to be treated and to the route of administration. The severity of the condition may, for example, be evaluated, in part, by standard prognostic evaluation methods. Further, the dose and perhaps dose frequency, will also vary according to the age, body weight, and response of the individual patient. A program comparable to that discussed above may be used in veterinary medicine.

Although the exact dosage will be determined on a drug-by-drug basis, in most cases, some generalizations regarding the dosage can be made. The daily dosage regimen for an adult human patient may be, for example, an oral dose of between 0.1 mg/m² and 2000 mg/m² body surface area per day of each active ingredient, typically between 1 mg/m2 and 500 mg/m2 body surface area per day, for example 5 mg/m² to 200 mg/m² body surface area per day. In other embodiments, an intravenous, subcutaneous, or intramuscular dose of each active ingredient of between 0.01 mg/m² and 100 mg/m² body surface area per day, typically between 0.1/m² mg and 60 mg/m² body surface area per day, for example, 1 mg/m² to 40 mg/m² body surface area per day can be used. In cases of administration of a pharmaceutically acceptable salt, dosages may be calculated as the free base. In some embodiments, the composition is administered 1 to 4 times per day. Alternatively the compositions of the invention may be administered by continuous intravenous infusion, preferably at a dose of each active ingredient up to 1000 mg/m² body surface area per day.

As will be understood by those of skill in the art, in certain situations it may be necessary to administer the compounds disclosed herein in amounts that exceed, or even far exceed, the above-stated, preferred dosage range in order to effectively and aggressively treat particularly aggressive diseases or infections. In some embodiments, the compounds will be administered for a period of continuous therapy, for example for a week or more, or for months or years.

Dosage intervals can also be determined using MEC value. Compositions should be administered using a regimen which maintains plasma levels above the MEC for 10-90% of the time, typically between 30-90% and most typically between 50-90%. In cases of local administration or selective uptake, the effective local concentration of the drug may not be related to plasma concentration.

The pharmaceutical compositions of the present invention may be manufactured in a manner that is itself known, e.g., by means of conventional mixing, dissolving, granulating, dragee-making, levigating, emulsifying, encapsulating, entrapping or tabletting processes.

Compounds disclosed herein can be evaluated for efficacy and toxicity using known methods. For example, the toxicology of a particular compound, or of a subset of the compounds, sharing certain chemical moieties, may be established by determining in vitro toxicity towards a cell line, such as a mammalian, and preferably human, cell line. The results of such studies are often predictive of toxicity in animals, such as mammals, or more specifically, humans. Alternatively, the toxicity of particular compounds in an animal model, such as mice, rats, rabbits, or monkeys, may be determined using known methods. The efficacy of a particular compound may be established using several recognized methods, such as in vitro methods, animal models, or human clinical trials. Recognized in vitro models exist for nearly every class of condition, including but not limited to cancer, cardiovascular disease, and various immune dysfunction. Similarly, acceptable animal models may be used to establish efficacy of chemicals to treat such conditions. When selecting a model to determine efficacy, the skilled artisan can be guided by the state of the art to choose an appropriate model, dose, and route of administration, and regime. Of course, human clinical trials can also be used to determine the efficacy of a compound in humans.

Specific Pharmaceutical Formulations

Injectables can be prepared in conventional forms, either as liquid solutions or suspensions, solid forms suitable for solution or suspension in liquid prior to injection, or as emulsions. Suitable excipients are, for example, water, saline, dextrose, mannitol, lactose, lecithin, albumin, sodium glutamate, cysteine hydrochloride, and the like. In addition, if desired, the injectable pharmaceutical compositions may contain minor amounts of nontoxic auxiliary substances, such as wetting agents, pH buffering agents, and the like. Physiologically compatible buffers include, but are not limited to, Hanks's solution, Ringer's solution, or physiological saline buffer. If desired, absorption enhancing preparations (for example, liposomes), may be utilized.

For transmucosal administration, penetrants appropriate to the barrier to be permeated may be used in the formulation. Pharmaceutical formulations for parenteral administration, e.g., by bolus injection or continuous infusion, include aqueous solutions of the active compounds in water soluble form. Additionally, suspensions of the active compounds may be prepared as appropriate oily injection suspensions. Suitable lipophilic solvents or vehicles include fatty oils such as sesame oil, or other organic oils such as soybean, grapefruit or almond oils, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents that increase the solubility of the compounds to allow for the preparation of highly concentrated solutions. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents. Alternatively, the active ingredient may be in powder form for constitution with a suitable vehicle, e.g., sterile pyrogen-free water, before use. For oral administration, the compounds can be formulated readily by combining the active compounds with pharmaceutically acceptable carriers well known in the art. Such carriers enable the compounds of the invention to be formulated as tablets, pills, dragees, capsules, liquids, gels, syrups, slurries, suspensions and the like, for oral ingestion by a patient to be treated. Pharmaceutical preparations for oral use can be obtained by combining the active compounds with solid excipient, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). If desired, disintegrating agents may be added, such as the cross-linked polyvinyl pyrrolidone, agar, or alginic acid or a salt thereof such as sodium alginate. Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures.

Pharmaceutical preparations which can be used orally include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. All formulations for oral administration should be in dosages suitable for such administration. For buccal administration, the compositions may take the form of tablets or lozenges formulated in conventional manner.

For administration by inhalation, the compounds for use according to the present invention are conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of, e.g., gelatin for use in an inhaler or insufflator may be formulated containing a powder mix of the compound and a suitable powder base such as lactose or starch.

The compounds may also be formulated in rectal compositions such as suppositories or retention enemas, e.g., containing conventional suppository bases such as cocoa butter or other glycerides.

In addition to the formulations described previously, the compounds may also be formulated as a depot preparation. Such long acting formulations may be administered by implantation (for example subcutaneously or intramuscularly) or by intramuscular injection. Thus, for example, the compounds may be formulated with suitable polymeric or hydrophobic materials (for example as an emulsion in an acceptable oil) or ion exchange resins, or as sparingly soluble derivatives, for example, as a sparingly soluble salt. For hydrophobic compounds, a suitable pharmaceutical carrier may be a cosolvent system comprising benzyl alcohol, a nonpolar surfactant, a water-miscible organic polymer, and an aqueous phase. A common cosolvent system used is the VPD co-solvent system, which is a solution of 3% w/v benzyl alcohol, 8% w/v of the nonpolar surfactant Polysorbate 80™, and 65% w/v polyethylene glycol 300, made up to volume in absolute ethanol. Naturally, the proportions of a co-solvent system may be varied considerably without destroying its solubility and toxicity characteristics. Furthermore, the identity of the co-solvent components may be varied: for example, other low-toxicity nonpolar surfactants may be used instead of POLYSORBATE 80™; the fraction size of polyethylene glycol may be varied; other biocompatible polymers may replace polyethylene glycol, e.g., polyvinyl pyrrolidone; and other sugars or polysaccharides may substitute for dextrose.

Alternatively, other delivery systems for hydrophobic pharmaceutical compounds may be employed. Liposomes and emulsions are well known examples of delivery vehicles or carriers for hydrophobic drugs. Certain organic solvents such as dimethylsulfoxide also may be employed, although usually at the cost of greater toxicity. Additionally, the compounds may be delivered using a sustained-release system, such as semipermeable matrices of solid hydrophobic polymers containing the therapeutic agent. Various sustained-release materials have been established and are well known by those skilled in the art. Sustained-release capsules may, depending on their chemical nature, release the compounds for a few weeks up to over 100 days. Depending on the chemical nature and the biological stability of the therapeutic reagent, additional strategies for protein stabilization may be employed.

The compositions may, if desired, be presented in a pack or dispenser device which may contain one or more unit dosage forms containing the active ingredient. The pack may for example comprise metal or plastic foil, such as a blister pack. The pack or dispenser device may be accompanied by instructions for administration. The pack or dispenser may also be accompanied with a notice associated with the container in form prescribed by a governmental agency regulating the manufacture, use, or sale of pharmaceuticals, which notice is reflective of approval by the agency of the form of the drug for human or veterinary administration. Such notice, for example, may be the labelling approved by the U.S. Food and Drug Administration for prescription drugs, or the approved product insert. Compositions comprising a compound of the invention formulated in a compatible pharmaceutical carrier may also be prepared, placed in an appropriate container, and labelled for treatment of an indicated condition.

As used herein, the terms “administering” and “administration” refer to any method of providing a pharmaceutical preparation to a subject. Such methods are well known to those skilled in the art and include, but are not limited to, oral administration, transdermal administration, administration by inhalation, nasal administration, topical administration, intravaginal administration, ophthalmic administration, intraaural administration, intracerebral administration, rectal administration, and parenteral administration, including injectables such as intravenous administration, intra-arterial administration, intramuscular administration, and subcutaneous administration. Administration can be continuous or intermittent. In various aspects, a preparation can be administered therapeutically; that is, administered to treat an existing disease or condition. In further various aspects, a preparation can be administered prophylactically; that is, administered for prevention of a disease or condition. In one aspect, administration of a tablet refers to oral administration.

As used herein, the term “immediate release” refers to the attribute indicating that a desired substance is released to its target environment relatively immediately. In one aspect, an “immediate release” tablet releases more than about 40% of the desired substance within hour following administration, as measured under the Tablet Dissolution Test.

As used herein, the term “controlled release” refers to the attribute indicating that a desired substance, such as a drug (e.g., a magnesium salt), is released to its target environment (e.g., a subject) in a controlled fashion, rather than immediately. Thus, a “controlled release” formulation releases no more than about 40% of the desired substance within 1 hour following administration, as measured under the Tablet Dissolution Test. “Controlled release” includes both “delayed release” and “sustained release” formulations. In one aspect, “controlled release” excludes “immediate release” formulations; however, it is contemplated that certain “controlled release” formulations can include an immediate release aspect. For example, a formulation having an immediate release control core and an enteric coating would not be referred to as an “immediate release” formulation; such a formulation can be referred to as a “controlled release” formulation and a “delayed release” formulation, but not as a “sustained release” formulation. Examples of a “controlled release” tablet include a “delayed release” tablet, a “sustained release” tablet, and a “delayed/sustained release” tablet.

As used herein, the term “delayed release” refers to the attribute indicating that a desired substance, such as a drug (e.g., a magnesium salt), is released to its target environment (e.g., a subject) at a time other than promptly after administration. In one aspect, the dosage form controls the drug release rate into the gastrointestinal tract, releasing the bulk of the drug in a portion of the gastrointestinal tract distal to the duodenum. This can decrease the incidence or severity of gastrointestinal side effects. Additionally, this can increase the amount of drug absorbed into the blood. In a further aspect, a “delayed release” formulation releases no more than about 5% of the desired substance within 2 hours following administration. In a yet further aspect, a “delayed release” formulation releases no more than about 5% of the desired substance within 2 hours following administration and releases no more than about 40% of the desired substance within 3 hours following administration. In an even further aspect, a “delayed release” formulation releases no more than about 5% of the desired substance within 2 hours following administration, no more than about 40% of the desired substance within 3 hours following administration, and no more than about 80% of the desired substance within 8 hours following administration. In an even further aspect, a “delayed release” formulation releases no more than about 5% of the desired substance within 2 hours following administration, no more than about 40% of the desired substance within 4 hours following administration, and from about 50 to about 80% of the desired substance within 8 hours following administration. In a further aspect, substantially the entire drug is released within 12 hours. “Delayed release” is a subset of “controlled release.” FDA guidelines also refer to a “delayed release” tablet as a solid dosage form, which releases a drug (or drugs) at a time other than promptly after administration. Enteric-coated articles are delayed release dosage forms. The term includes both “delayed release” tablets and “delayed/sustained release” tablets.

As used herein, the term “sustained release” refers to the attribute indicating that a desired substance, such as a drug (e.g., a magnesium salt), is released to its target environment (e.g., a subject) in a desired dosage, which is maintained over a desired interval. In one aspect, this attribute can be also referred to as “extended release” or “prolonged release.” In one aspect, the dosage form controls the drug release rate so as to decrease the frequency of dosing. This can maintain desired blood levels of the drug independent of dosing frequency. This can also increase patient compliance with a given treatment regimen. In a further aspect, the dosage form controls the drug release rate so as to target the distal small intestine. In a yet further aspect, the dosage form controls the drug release rate so as to target the distal small intestine, thereby increasing the amount of magnesium available for interaction with TRPM6 and/or TRPM7 cation channels. In a further aspect, a “sustained release” formulation releases no more than about 40% of the desired substance within 1 hour following administration. In a yet further aspect, a “sustained release” formulation releases no more than about 40% of the desired substance within 1 hour following administration, and no more than about 80% of the desired substance within 6 hours following administration. In an even further aspect, a “sustained release” formulation releases no more than about 40% of the desired substance within 1 hour following administration, and from about 50% to about 80% of the desired substance within 6 hours following administration. In a further aspect, substantially the entire drug is released within 10 hours. In a still further aspect, a “sustained release” formulation releases no more than about 5% of the desired substance within 2 hours following administration and releases no more than about 40% of the desired substance within 3 hours following administration. In an even further aspect, a “sustained release” formulation releases no more than about 5% of the desired substance within 2 hours following administration, no more than about 40% of the desired substance within 3 hours following administration, and no more than about 80% of the desired substance within 8 hours following administration. In an even further aspect, a “sustained release” formulation releases no more than about 5% of the desired substance within 2 hours following administration, no more than about 40% of the desired substance within 3 hours following administration, and from about 50% to about 80% of the desired substance within 8 hours following administration. In a further aspect, substantially all of the entire drug is released within 12 hours. “Sustained release” is a subset of “controlled release.” FDA guidelines also refer to a “sustained release” tablet as an “extended release tablet”—that is, a solid dosage form containing a drug which allows at least a reduction in dosing frequency as compared to that drug presented in conventional dosage form. The term includes both “sustained release” tablets and “delayed/sustained release” tablets.

Materials and Methods

Cell Culture and Treatments:

HepAD38.7-Tet cells used in this study was cultured in HyClone DMEM media supplemented with 1% Pen Strep, 10% tetracycline-free FBS (Biowest), 400 μg/mL G418 and/or 5 μg/mL tetracycline. PHH were purchased from BioreclamationlVT (product number: UNR-M00995-P) and were recovered according to the BioIVT protocol. The cells were maintained in Williams' medium E containing B27, Glutamax (Gibco) and Pen Strep (Gibco) and supplemented with 5C condition: 20 μM of Forskolin, 10 μM of B431542, 0.5 μM IWP2, 5 μM DAPT and 0.1 μMLDN193189 as described.

Oligos, siRNA. Oligonucleotides used in this study were designed using Primer Plus and synthesized by Integrated DNA technologies, Inc (IDT). Validated siRNAs targeting 3 different regions of the 69 genes were ordered from Silencer Select (Applied Biosystems).

HBV infection of PHH. Primary human hepatocytes were infected at 200 genome equivalents of 200 in presence of 4% PEG8000 and 1% DMSO as described earlier. The samples, cell lysate for RNA and or DNA as well as cell culture supernatant were harvested at indicated timepoints on figure legends.

RNAi screening in HepAD38.7 cells. HepAD38.7 cells were maintained as described earlier. A pool of three siRNA per gene was spotted in 384 well plates at a final concentration of 20 nM. Gene silencing was carried out by reverse transfection as per the Lipofectamine™ 3000 reagent protocol (0.1 μL Lipofectamine 3000 per well in Opti-MEM medium) (ThermoScientific). The cells were washed, and the media changed one-day post-transfection. The transfected cells were incubated at 37° C., 5% C02 incubator for 72 hours. For reproducibility, the screen was repeated independently three times. Four technical replicates were included in the plate, and 2 biological replicates were included for each screen. After three days of incubation, the cell culture supernatant was harvested for HBsAg ELISA. In order to harvest enough samples for both RNA, DNA extraction as well as ELISA, a follow-up screen was carried out in an upscale format of 24 and 12 wells.

HBsAg ELISA. HBsAg ELISA was carried out as described in detail below. In brief 25 μL of the cell culture supernatant was collected at the indicated timepoints on the figure legends and was used for the HBsAg ELISA.

Reagents:

Streptavidin-HRP Elisa 1 ml, Cat #(421)554066—BD Bioscience/Zuellig Pharma

TMB Substrate OptEIA Reagent Set, Cat #(421)555214—BD Bioscience/Zuellig Pharma

CAP Carbonate-Bicarbonate Buffer Capsules, Cat #C3041-100—TR Sigma Aldrich

384 well polystyrene plate maxisorp, Clear, Cat #P6366-1 CS-Sigma Aldrich

Mouse monoclonal [86c] to Hepatitis B Virus Surface Antigen (Ad/Ay), Cat #ab20758—Abcam

Rabbit polyclonal to Hepatitis B Virus Surface Antigen (Ad/Ay) (Biotin), Cat #ab68520—Abcam

Active Hepatitis B Surface Antigen (Adw) full length protein, Cat #ab91276—Abcam

Tween 20

Wash Buffer: 0.05% Tween 20 in 1×PBS

Assay diluent: 10% FBS in 1×PBS

Procedure
1. Coat microwells with 25 ul per well of mouse monoclonal [86C] diluted in coating buffer (6 ug per ml; 300 ul from 0.2 mg/ml stock in 10 ml of coating buffer)(150 ng/well).
2. Seal plate and incubate overnight at 4° C.
3. Aspirate wells and wash 3 times with 50 ul/well of wash buffer.
4. After last wash, invert plate and blot on c-towel and spin 1000 rpm for 1 min to remove residual solution.
5. Block plates with 75 ul/well of assay diluent. Incubate at RT for 2 hrs.
6. Aspirate/wash as in step 3.
7. Prepare 4000 ng/ml of active HBS Ag (Adw) full length protein in assay diluent and titrate 2 fold for 12 different decreasing concentration. Pipette 25 ul of sample and different dilutions of active HepB surface Ag (Adw) into appropriate wells. Seal plate and incubate for 2 hrs at RT.
8. Aspirate/wash as in step 3 but with total 5 washes.
9. Add 25 ul of working detector (Rabbit polyclonal to HBS-Biotin (4500 ng/ml)+SAv-HRP (1:1000) reagent to each well. Seal plate and incubate for 1 hr at RT.
10. Aspirate/wash as in step 3 but for 7 washes.
11. Add 25 ul of TMB substrate solution to each well and incubate plate for 30 minutes at RT in dark.
12. Add 25 ul of stop solution (1N HCl) to each well, read absorbance at 450 nm within 30 minutes of stopping reaction.

Quantitative Real-Time PCR. RNA was extracted from cell lysates according to the Total RNeasy kit protocol (Qiagen). Pre-genomic RNA was detected using SYBR Fast based RT-PCR kit in the Quantstudio 7 PCR system (Applied Biosystems).

pgRNA Primers Used:

forward:
(SEQ ID No. 1)
CGTTTTTGCCTTCTGACTTCTTTC
and
reverse:
(SEQ ID No. 2)
ACAGAGCTGAGGCGGTGTCTA.

HBV extracellular DNA was extracted from 200 μL of cell culture supernatant collected at the end of the experiment as indicated on the figure legends. DNA was extracted according to the QIAamp DNA Mini Kit (Qiagen).

HBV DNA Primers Used for the Detection of Extracellular DNA:

forward:
(SEQ ID No. 3)
CCGTCTGTGCCTTCTCATCTG
and
reverse:
(SEQ ID No. 4)
AGTCCAAGAGTCCTCTTATGTAAGACCTT.

All primers used in this study for quantitative polymerase chain reaction (qPCR) were purchased from Integrated DNA Technologies. qPCR data were analyzed using the Livack method as described.

Western blot analysis. At the end of the experiment, the cells were harvested in radioimmunoprecipitation assay (RIPA) buffer (Cat No: 89900, ThermoScientific) supplemented with 1× protease and phosphatase inhibitor cocktail (Roche). The lysates were further incubated on ice for 30 minutes and thereafter clarified by centrifugation at 14,000×g for 15 minutes at 4° C. The supernatant was harvested, and the protein quantified by bicinchoninic acid protein assay (ThermoScientific, cat no: 23225) according to the manufacturer's protocol. 30 μg of proteins were boiled at 95° C. for 5 minutes and separated by SDS-polyacrylamide gel electrophoresis. The separated proteins were thereafter transferred to PVDF membranes, blocked with Intercept™ (TBS) buffer (Bio-Rad, P/N: 927-70001) or 5% skim milk for 1 hour at room temperature and incubated with primary antibodies at 4° C. overnight. The membranes were washed three times with 1×TBST and incubated with LI-COR IRDye® 8000W or 680CW antibodies at room temperature for 1 hour. The membranes were washed five times with 1×TBST and visualized using ChemiDoc XRS+system (Bio-Rad).

Statistical analysis. Data analysis was carried out in GraphPad prism 8 using different tests as indicated in the figure legends. Network analysis of the upregulated genes was carried on STRING database as described. Differentially expressed genes from the RNAseq data was analyzed in R studio using DEGseq package as reported.

FIG. 2 shows a workflow or method 200 to identify hit compounds for treating Hepatitis B and development into a possible candidate for use to treat Hepatitis B. In particular it shows the high throughput screening of epigenetic modulators, determination of HITS based on high HBsAg reduction and low cytotoxicity, validation in HepAD8.7 (an expression system for HBV replication) and in live infection models (HepG2-NTCP cell line and primary human hepatocytes). In block 205, a primary screen of the library of compounds was done in HepG2 2.15 cells to determine the inhibitory activity of HBsAg (the surface antigen of the Hepatitis B virus which indicates hepatitis B infection in patients) and parallel cytotoxicity. In block 310, the hit compounds (“HITS” or “HITs”) were determined by identifying compounds with certain criteria. For example, activity greater than 50% (i.e. greater than 50% inhibition), and optionally compounds with less than 20% cytotoxicity or about 20% cytotoxicity. In the preliminary screen, the compounds are typically tested at higher concentrations than expected and may lead to greater than desired cytotoxicity (>20%). For example, the compounds may be tested at 3 μM (data shown herein) or 10 μM. However, the compound was subsequently further tested in a proper dose response manner (described below) to determine whether the drug is able to elicit the antiviral response (decrease in HBsAg levels) with low cytotoxicity (i.e. <20% cytotoxicity).

In block 215, a secondary screen was conducted to determine the EC50 values (the effective concentration to obtain 50% activity or inhibition) of the HIT compounds. In block 220, the hit compounds were tested in HEPAD38.7 HBV live infection models to determine the robustness of the hit compounds, i.e. whether the hit compounds are effective. In block 225, the novel lead compounds or scaffold structures are identified and mechanism of action (MOA) studies for target engagement were developed. In block 230, the lead compound is optimised and validated or if the compound is known but for a different non-biological use or biological target, the compound may be repurposed for the inhibition of HBV. In block 235, the efficacy of the compound was determined in animal models.

A library of compounds was used in the primary screen. The library contains inhibitors of the following enzymes: histone deactylases (HDACs), sirtuins (SIRTs), histone methyl transferases (HMTs), DNA methyltransferases as well as SIRT activators. The inhibitors have a variety of structurally and mechanistically different compound classes.

The library may be made up of a commercially available library from Enzo containing 42 compounds and a commercially available library from Selleckchem containing 151 compounds. These compounds have known activity against enzymes which carry out epigenetic modifications (as above). These libraries are a useful tool for chemical genomics, assay development and other pharmacological applications.

In addition, the library may further contain the Micro Source—Spectrum collection, which is a collection of bioactive compounds and natural products, and contains about 2400 compounds and include all of the compounds in the US and International Drug Collections, together with MicroSource Natural Product and Discover libraries.

From the primary high throughput screen of 151 epigenetic modulators in the SelleckChem compound library at a 3 μM concentration, 16 hit compounds were identified to have good antiviral activity resulting in high HBsAg loss (>50%) and low cytotoxicity (<20%) and the results are shown in FIG. 3. The high throughput screen had a hit rate of approximately 10%.

Table 1 below shows the hit compounds identified, their known inhibition activity, and chemical structure.

TABLE 1
HIT compounds

| S/No. | Identifier | Inhibitor Type | Structure |
|---|---|---|---|
| 1 | AZ960 | JAK2 | 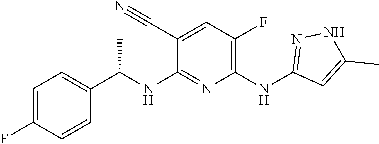 [SMILES: Cc1cc(Nc2nc(N[C@@H](C)c3ccc(F)cc3)c(C#N)cc2F)n[nH]1] |
| 2 | CYC116 | Aurora kinase A/B (AURK A/B) | 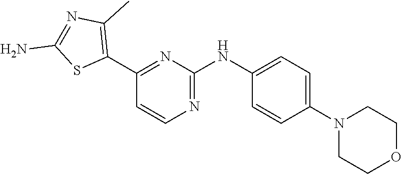 [SMILES: Cc1nc(N)sc1-c1ccnc(Nc2ccc(N3CCOCC3)cc2)n1] |
| 3 | MI-3 | Menin-MLL | 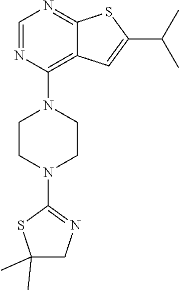 [SMILES: CC(C)c1cc2c(N3CCN(C4=NCC(C)(C)S4)CC3)ncnc2s1] |
| 4 | Nexturastat A | HDAC6 | 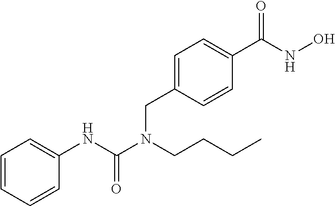 [SMILES: CCCCN(Cc1ccc(C(=O)NO)cc1)C(=O)Nc1ccccc1] |
| 5 | TAK-901 | Aurora kinase A/B (AURK A/B) | 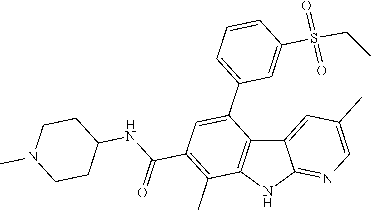 [SMILES: CCS(=O)(=O)c1cccc(-c2cc(C(=O)NC3CCN(C)CC3)c(C)c3[nH]c4ncc(C)cc4c23)c1] |
| 6 | Tubastatin A hydrochloride salt | HDAC6 | 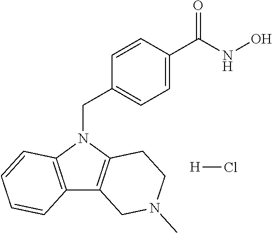 |
| 7 | Alisertib | Aurora A | 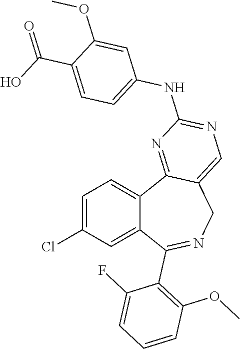 [SMILES: COc1cc(Nc2ncc3c(n2)-c2ccc(Cl)cc2C(c2c(F)cccc2OC)=NC3)ccc1C(=O)O] |
| 8 | MC-1568 | HDAC | 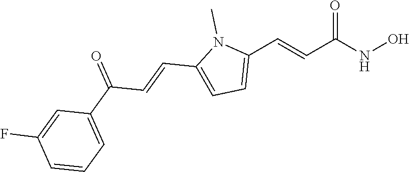 [SMILES: Cn1c(/C=C/C(=O)NO)ccc1/C=C/C(=O)c1cccc(F)c1] |
| 9 | MI-2 | Menin-MLL | 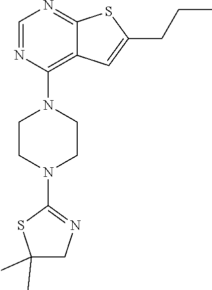 [SMILES: CCCc1cc2c(N3CCN(C4=NCC(C)(C)S4)CC3)ncnc2s1] |
| 10 | OF-1 | BRPF1B and BRPF2 (BRD1) | 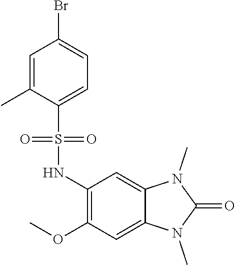 [SMILES: COc1cc2c(cc1NS(=O)(=O)c1ccc(Br)cc1C)n(C)c(=O)n2C] |
| 11 | Resveratrol | Wide-spectrum including SIRT | 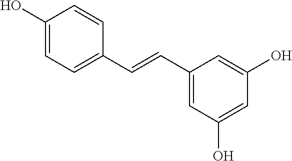 [SMILES: Oc1ccc(/C=C/c2cc(O)cc(O)c2)cc1] |
| 12 | Tofacitinib | JAK2/3 | 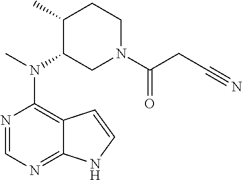 [SMILES: C[C@@H]1CCN(C(=O)CC#N)C[C@@H]1N(C)c1ncnc2[nH]ccc12] |
| 13 | Tubastatin A | HDAC6 | 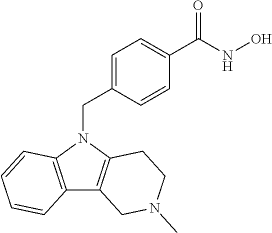 [SMILES: CN1CCc2c(c3ccccc3n2Cc2ccc(C(=O)NO)cc2)C1] |
| 14 | EX-527 | SIRT1 | 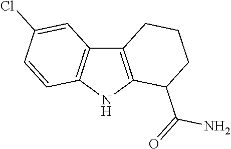 [SMILES: NC(=O)C1CCCc2c1[nH]c1ccc(Cl)cc21] |
| 15 | TMP-269 | HDAC 4/5/7/9 | 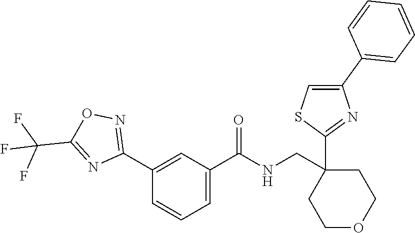 [SMILES: O=C(NCC1(c2nc(-c3ccccc3)cs2)CCOCC1)c1cccc(-c2noc(C(F)(F)F)n2)c1] |
| 16 | UPF1069 | PARP | 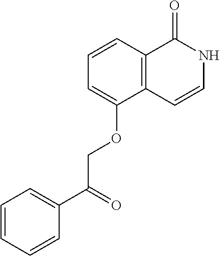 [SMILES: O=C(COc1cccc2c(=O)[nH]ccc12)c1ccccc1] |

A secondary screen of the hit compounds was done to determine the EC50 of the hit compounds. FIG. 4 shows the 8-point dose response curves of the compounds in HepG2 2.15 cells starting from 30 μM. The lighter coloured curve or line (upper curve) shows the % HBsAg inhibition while the darker coloured curve or line (lower curve) shows the cytotoxicity. All 16 compounds showed HBsAg inhibition. However, it may be observed that only AZ960, CYC116, MI-3, TAK-901, Tubastatin A hydrochloride salt, and Nexturastat A showed a suitable dose response curve (i.e. the % inhibition changes with the dose). Some of the other compounds were determined to be less suitable. For example, alisertib was not as efficacious in reducing HBsAg levels and also did not show an effect on HepAD38 cells. The MI-2 EC50 is very high and appers to be dependent on the toxic nature of the compound and was not a viable option. The percentage inhibition curve of MC1568, OF-1 and tofacitinib in FIG. 4 are close to 0 in the vertical axis and may not be readily seen in FIG. 4. It may be seen that the secondary screen is able to filter off unsuitable compounds from the primary screen.

In FIG. 5, a therapeutic window for the six most promising compounds—AZ960, CYC116, MI-3, TAK-901, Tubastatin A hydrochloride salt, and Nexturastat A are shown. The therapeutic window is defined as the concentrations of the compound between the EC50 of the compound and 50% cytotoxicity is reached. The IC50 values of the HBsAg inhibition and cytotoxicity for these 6 compounds are provided in Table 2 and shows numerically the therapeutic window. Two of the compounds were Aurora Kinase inhibitors (TAK-901 and CYC-116), two of the compounds were inhibitors to HDAC6 (Nexturastat A and Tubastatin A HCl), one compound was a known inhibitor of the Menin-MLL interaction (MI-3) and a final one was an inhibitor to JAK-2 (AZ960).

TABLE 2
IC50 values for HBsAg inhibition and cytotoxicity in Hep G2 2.15 cells

| | μM | |
|---|---|---|
| Inhibitor | IC50 (HBsAg) | IC50 (Cytotoxicity) |
| TAK-901 | 0.3996 | 10.97 |
| CYC-116 | 0.42 | 1.486 |
| AZ960 | 0.1934 | 0.2309 |
| MI-3 | 1.122 | 4.372 |
| Nexturastat A | 0.98 | 3.582 |
| Tubastatin A HCl | 2 | 7.211 |

Based on these six compounds, possible mechanistic targets were identified based on the annotated targets previously—JAK2, Aurora Kinase A and B (AURKA and AURKB), Menin-MLL interaction, and HDAC6. siRNAs for each of these were designed to probe their effects on HBV.

A secondary validation of the drug screen in HepAD38.7 cells and a functional gene knockdown using Lipofectamine 3000 reagent was also performed. The genes selected were the annotated targets of the selected epigenetic inhibitors.

FIG. 6 shows the experimental setup to culture HepAD38.7 cells and treatment with small interfering RNA (siRNAs). Tetracycline-dependent viral induction is used in this method. The HBV is maintained in the “Virus On” condition and seeded in a 24 well plate with the cells. On the next day, different siRNAs (sequences shown in Table 3) were individually added including siAURKA, siAURKB, siMenin1, siHDAC6, siJAK2, as well as a siRNA negative control. After 3 days, the supernatant is collected for use with the HBsAg ELISA assay and DNA isolation for eHBV analysis.

The cells are fixed in 4% paraformaldehyde and permeabilized with 0.2% Triton X 100 and stained with HBcAg antibody to determine the antigen levels by IFA.

The DNA is isolated from the cellular lysate, purification with Plasmid safe ATP-dependent DNAse is performed to remove non-circular DNAs (relaxed circular DNA). cccDNA levels are then measure using a Taqman-probe based quantitative polymerase chain reaction.

TABLE 3
Silencer select human siRNA sequences used for the functional knock-
down in both HepAD38.7 cell s as well as primary human hepatocytes

| Sample ID | ASX20238 | ASX203GN | ASX204PT | ASX208HU |
|---|---|---|---|---|
| RefSeq | NM_198437 | NM_004217 | NM_006044 | NM_130801 |
| Accession | | | | |
| Number | | | | |
| Gene Symbol | AURKA | AURKB | HDAC6 | MEN1 |
| Full Gene | aurora kinase | aurora kinase | histone | multiple |
| Name | A | B | deacetylase 6 | endocrine |
| | | | | neoplasia I |
| Gene ID | 6790 | 9212 | 10013 | 4221 |
| siRNA ID A | s195 | s17611 | s19459 | s8682 |
| siRNA ID B | s196 | s17612 | s19461 | s8683 |
| siRNA ID C | s197 | s17613 | s19460 | s8684 |

| | |
|---|---|
| siRNA A sequences | S: GCGCAUUCCUUUGCAAGCATT |
| | A/S: UGCUUGCAAAGGAAUGCGCTG |
| siRNA B sequences | S: GAGUCUACCUAAUUCUGGATT |
| | A/S: UCCAGAAUUAGGUAGACUCTG |
| siRNA C sequences | S: GGAUCAGCUGGAGAGCUUATT |
| | A/S: UAAGCUCUCCAGCUGAUCCAA |

| | |
|---|---|
| siRNA A sequences | S: CCUGCGUCUCUACAACUAUTT |
| | A/S: AUAGUUGUAGAGACGCAGGAT |
| siRNA B sequences | S: UCGUCAAGGUGGACCUAAATT |
| | A/S: UUUAGGUCCACCUUGACGATG |
| siRNA C sequences | S: GCAAGUUUGGAAACGUGUATT |
| | A/S: UACACGUUUCCAAACUUGCCT |

| | |
|---|---|
| siRNA A sequences | S: CCGUGAGAGUUCCAACUUUTT |
| | A/S: AAAGUUGGAACUCUCACGGTG |
| siRNA B sequences | S: CAGUUUAUCUGCAUCCGAATT |
| | A/S: UUCGGAUGCAGAUAAACUGAG |
| siRNA C sequences | S: GGAGGGUCCUUAUCGUAGATT |
| | A/S: UCUACGAUAAGGACCCUCCGG |

| | |
|---|---|
| siRNA A sequences | S: GAAGGUCUCCGAUGUCAUATT |
| | A/S: UAUGACAUCGGAGACCUUCTT |
| siRNA B sequences | S: GACCUACUAUCGGGAUGAATT |
| | A/S: UUCAUCCCGAUAGUAGGUCTT |
| siRNA C sequences | S: CCAUUGACCUGCACACCGATT |
| | A/S: UCGGUGUGCAGGUCAAUGGAA |

SEQ ID No. 5:
AURKA siRNA A sequence
S: GCGCAUUCCUUUGCAAGCATT
SEQ ID No. 6:
AURKA siRNA A sequence
NS: UGCUUGCAAAGGAAUGCGCTG
SEQ ID No. 7:
AURKA siRNA B sequence
S: GAGUCUACCUAAUUCUGGATT
SEQ ID No. 8:
AURKA siRNA B sequence
NS: UCCAGAAUUAGGUAGACUCTG
SEQ ID No. 9:
AURKA siRNA C sequence
S: GGAUCAGCUGGAGAGCUUATT
SEQ ID No. 10:
AURKA siRNA C sequence
NS: UAAGCUCUCCAGCUGAUCCAA
SEQ ID No. 11:
AURKB siRNA A sequence
S: CCUGCGUCUCUACAACUAUTT
SEQ ID No. 12:
AURKB siRNA A sequence
NS: AUAGUUGUAGAGACGCAGGAT
SEQ ID No. 13:
AURKB siRNA B sequence
S: UCGUCAAGGUGGACCUAAATT
SEQ ID No. 14:
AURKB siRNA B sequence
NS: UUUAGGUCCACCUUGACGATG
SEQ ID No. 15:
AURKB siRNA C sequence
S: GCAAGUUUGGAAACGUGUATT
SEQ ID No. 16:
AURKB siRNA C sequence
NS: UACACGUUUCCAAACUUGCCT
SEQ ID No. 17:
HDAC6 siRNA A sequence
S: CCGUGAGAGUUCCAACUUUTT
SEQ ID No. 18:
HDAC6 siRNA A sequence
NS: AAAGUUGGAACUCUCACGGTG
SEQ ID No. 19:
HDAC6 siRNA B sequence
S: CAGUUUAUCUGCAUCCGAATT
SEQ ID No. 20:
HDAC6 siRNA B sequence
NS: UUCGGAUGCAGAUAAACUGAG
SEQ ID No. 21:
HDAC6 siRNA C sequence
S: GGAGGGUCCUUAUCGUAGATT
SEQ ID No. 22:
HDAC6 siRNA C sequence
NS: UCUACGAUAAGGACCCUCCGG
SEQ ID No. 23:
MEN1 siRNA A sequence
S: GAAGGUCUCCGAUGUCAUATT
SEQ ID No. 24:
MEN1 siRNA A sequence
NS: UAUGACAUCGGAGACCUUCTT
SEQ ID No. 25:
MEN1 siRNA B sequence
S: GACCUACUAUCGGGAUGAATT
SEQ ID No. 26:
MEN1 siRNA B sequence
NS: UUCAUCCCGAUAGUAGGUCTT
SEQ ID No. 27:
MEN1 siRNA C sequence
S: CCAUUGACCUGCACACCGATT
SEQ ID No. 28:
MEN1 siRNA C sequence
NS: UCGGUGUGCAGGUCAAUGGAA

FIG. 7 shows the results of the functional knockdown of each of AURKA, AURKB, Menin1, HDAC6 and JAK2 in the HepAD38.7 cells by the siRNAs. The siRNAs cause the HBsAg levels and eHBV DNA levels to be lower than the negative control and control samples. In particular, as shown by the left chart in FIG. 7 the HBsAg levels measured by ELISA upon genetic knockdown of the target genes in HepAD38.7 cells shows good reduction in AURKB and HDAC6 gene. The right chart with the extracellular HBV DNA levels in the supernatant shows the highest reduction in AURKB knockdown. From these data, it may be seen that the knockdown of AURKB causes the largest decrease in both HBsAg and eHBV DNA levels. The knockdown of JAK2 also causes a decrease in both measured HBV levels. However, the knockdown of the other three targets provide mixed results. This indicates that the knockdown of AURKA, AURKB, Menin1, HDAC6 and JAK2 results in decreased HBV activity, and proves that the knockdown of these genes could lead to potent antiviral effect. The AURK inhibitors (TAK-901 and CYC-116) and HDAC6 inhibitors (Nexturastat A and Tubastatin A HCl) showed significantly higher antiviral activity and were studied further.

FIG. 8 briefly describes an experimental model of the method to infect the cells with HBV, treatment with drugs, and subsequent analysis. The primary human hepatocytes were seeded in a 96 well plate coated with Collagen I (30 minutes at room temperature) with DMSO (1%) induction. The next day, the cells were infected with purified HBV with a multiplicity of infection (MOI) of 500 (i.e. the ratio of the infection agent (HBV) to the infection target (cell)) in media containing 4% polyethylene glycol 8000 (PEG-8000) and 1% DMSO (day 0). On the next day, the infected cells were washed vigorously to remove excess virus by washing 3 times without removing the spent media completely and treated with the test compounds at 3 μM concentration (day 1). In addition. the supernatant was collected at day 3 and the cells pulsed again with the test compounds at 3 μM concentration. At day 5, the supernatant was collected for analysis with the HBsAg ELISA and finally at day 8 cells were lysed for DNA isolation, eHBV analysis and cccDNA analysis. This allows comparison of the effects of the compounds on day 3 and day 5. Some of the cells were fixed, permeabalized and stained with antibody to HBcAg to determine HBcAg levels via IFA.

FIG. 9 shows the results of the treatment of the HIT compounds in infected primary human hepatocytes (PHHs) in comparison with Entecavir (an approved drug used to treat Hepatitis B clinically). At both Day 3 and Day 5, the results shows that TAK-901, CYC-116, Nexturastat A, and Tubastatin A HCl, reduces HBsAg levels more than Entecavir at the same dose. In particular, CYC-116 and Tubastatin A HCl shows the lowest HBsAg levels. The data of the HBsAg levels at day 3 and day 5 in primary human hepatocytes show a significant decline in all 4 drug treatments starting at day 3 and was sustained at day 5.

FIG. 10 shows that Nexturastat A and Tubastatin A HCl had similar low toxicity levels to Entecavir while TAK-901 and CYC-116 had higher cytotoxicity levels (determine using a cell counting kit-8 (CCK-8)). In particular, the cell cytotoxicity was between 20-30% in the AURKB inhibitors whereas the HDAC6 inhibitors had very low cytotoxicity in these PHHs FIG. 11 shows the Immunofluorescent imaging of HBcAg at day 8 after drug treatment. It may be seen that the six hit compounds all reduce the HBcAg levels in infected primary human hepatocytes comparable to Entecavir (ENTE). FIG. 11a shows the HBcAg levels in the infected control with E-cadherin in the background. FIG. 11b show the HBcAg levels in the drug-treated cells at day 8. FIG. 11c shows the quantification data of HBcAg intensity in the middle panels FIG. 12 shows the effects of the HITs in the primary human hepatocytes on extracellular HBV DNA levels in the cell culture supernatant and cccDNA levels (determined by qPCR) in the cell lysate of the HBVcc-infected primary human hepatocytes. It may be seen that TAK-901, CYC-116 and AZ960 reduces extracellular HBV DNA levels more than Entecavir. TAK-901 and CYC-116 also provide the lowest measured cccDNA levels. It may be seen that the AURK and HDAC6 inhibitors appear to have the greatest effect based on both measurements.

FIG. 13 shows the results of the RNAi functional knockdown of candidate genes using Silencer Select siRNA and Lipofectamine 3000. It may be seen that there is little or no effect on albumin production meaning it does not negatively affect the host cell hepatocyte function but causes a decrease in HBV pregenomic RNA (pgRNA) levels as measured by qPCR. In particular, the pgRNA levels went down significantly in the AURKB and HDAC6 genetic knockdown. The pgRNA control sample means mock infected, i.e. no HBV is present hence the pgRNA measurement is nil as expected.

FIG. 14 shows that the knockdown of AURKA, AURKB. HDAC6, and MEN1 decreases the extracellular HBV DNA levels and cccDNA levels (as determined by qPCR) and thus inhibits the HBV in the infected primary human hepatocytes. In particular, there is a strong decline in the extracellular HBV DNA and cccDNA levels when AURK and HDAC6 are downregulated.

It will be appreciated that the knockdown of the different genes and proteins have differing effects on the various HBV biomarkers, thus all of these targets are possible targets alone or in combination to develop an effective treatment for Hepatitis B.

Based on the results obtained, it is believed that the knockdown or inhibition of the Aurora Kinase A and B (AURKA and AURKB), and HDAC6 provide the most likely (and/or most efficacious) target for treatment of Hepatitis B. It is also possible that the knockdown or inhibition of JAK2 and the Menin-MLL interaction may be an alternative target. Further investigation in to the role of AURK and HDAC6 in the viral life cycle is required. Without being bound by theory, it is known that HDAC6 induces deacetylation of Ac-Tubulin and destabilises the microtubule. It is believed that AURK and HDAC6 induces nuclear trafficking of other viruses and there is a functional association of microtubule in capsid assembly and viral replication. AURK and HDAC6 could be involved in the microtubule dynamics especially tubulin protein and affect the transport of HBV that entered the cell into the nucleus of the cell. Thus, it is possible that the inhibition or knockdown of AURK and HDAC could lead to decreased HBV activity by one or both of these mode of actions.

The HIT compounds may also be combined with other current treatment methods for Hepatitis B including in combination with currently used nucleoside analogues (NUCs) to treat Hepatitis B. Examples of nucleoside analogues that may be used include Entecavir, Tenofovir, Lamivudine, Adefovir, and Telbivudine or in combination with pegylated interferon (PEG-IFN) therapy as well. This includes any prodrug form or pharmaceutically acceptable salt of the nucleoside analogues. For example, Tenofovir exists in two prodrug forms commercially Tenofovr disoproxil and Tenofovir alafenamide. PEG-IFN therapy may be pegylated interferon alfa-2a or in combination with pegylated interferon-alpha-2b and pegylated interferon-beta-1a.

The results described herein shows that AURKA, AURKB, HDAC6, MEN1 (and hence the Menin-MLL interaction), and JAK2 are possible biological targets in the treatment of Hepatitis B, in particular AURKA, AURKB and HDAC6. Compounds that inhibit these biological targets or the functional knockdown of these biologically targets through the use of RNAi methods may be a possible treatment method of Hepatitis B.

1. A compound selected from the group consisting of AZ960, CYC116, MI-3, Nexturastat A, TAK-901, Tubastatin A hydrochloride salt, and a small interfering RNA molecule comprising any one of SEQ ID No. 5 to SEQ ID No. 28 for use in the treatment of Hepatitis B.

2. The compound according to claim 1, wherein the compound is selected from the group consisting of CYC116, Nexturastat A, TAK-901, Tubastatin A hydrochloride salt, and a small interfering RNA molecule comprising any one of SEQ ID No. 5 to SEQ ID No. 22.

3. Use of a compound selected from the group consisting of AZ960, CYC116, MI-3, Nexturastat A, TAK-901, Tubastatin A hydrochloride salt, and a small interfering RNA molecule comprising any one of SEQ ID No. 5 to SEQ ID No. 28 in the manufacture of a medicament for the treatment of Hepatitis B.

4. The use of the compound according to claim 3, wherein the compound is selected from the group consisting of CYC116, Nexturastat A, TAK-901, Tubastatin A hydrochloride salt, and a small interfering RNA molecule comprising any one of SEQ ID No. 5 to SEQ ID No. 22.

5. A method of treating a Hepatitis B infection in a subject, the method comprises administering to the subject a therapeutically effective dose of a compound selected from the group consisting of AZ960, CYC116, MI-3, Nexturastat A, TAK-901, Tubastatin A hydrochloride salt, and a small interfering RNA molecule comprising any one of SEQ ID No. 5 to SEQ ID No. 28,

6. The method according to claim 5, wherein the compound is selected from the group consisting of CYC116, Nexturastat A, TAK-901, Tubastatin A hydrochloride salt, and a small interfering RNA molecule comprising any one of SEQ ID No. 5 to SEQ ID No. 22.

7. The method according to any one of claims 5 to 6, further comprising administering a nucleoside analogue or pegylated interferon active against a Hepatitis B virus.

8. The method according to claim 7, wherein the nucleoside analogue is selected from the group consisting of entecavir, tenofovir, lamivudine, adefovir, telbivudine, and any prodrug and/or any pharmaceutically salt form of the compounds.

9. A composition comprising a compound selected from the group selected from the group consisting of AZ960, CYC116, MI-3, Nexturastat A, TAK-901, Tubastatin A hydrochloride salt, and a small interfering RNA molecule comprising any one of SEQ ID No. 5 to SEQ ID No. 28; and a nucleoside analogue or pegylated interferon active against a Hepatitis B virus.

10. The composition according to claim 9, wherein the compound is selected from the group consisting of CYC116, Nexturastat A, TAK-901, Tubastatin A hydrochloride salt, and a small interfering RNA molecule comprising any one of SEQ ID No. 5 to SEQ ID No. 22.

11. The composition according to any one of claims 9 to 10, wherein the nucleoside analogue is selected from the group consisting of entecavir, tenofovir, lamivudine, adefovir, and telbivudine.

12. The composition according to any one of claims 9 to 11, for use as a medicament.

13. The composition according to claim 12, for use in the treatment of Hepatitis B.

14. Use of the composition according to any one of claims 9 to 11 in the manufacture of a medicament for the treatment of Hepatitis B.
